(19) United States
(12) Patent Application Publication
(10) Pub. No.: US 2004/0063782 A1
(43) Pub. Date: Apr. 1, 2004

(54) Bicalutamide forms

(76) Inventor: Raymond J.H. Westheim, Nijmegen (NL)

Correspondence Address: MARK R. BUSCHER, P.O. BOX 161, CATHARPIN, VA, 20143, US

(21) Appl. No.: 10/660,775

(22) Filed: Sep. 12, 2003

Related U.S. Application Data
(60) Provisional application No. 60/413,765, filed on Sep. 27, 2002.
(60) Provisional application No. 60/470,223, filed on May 14, 2003.

(51) Int. Cl. A61K 31/277

(52) U.S. Cl. 514/522; 558/410

(57) ABSTRACT

A new crystalline form of bicalutamide (form II) is disclosed. Bicalutamide form II is useful as a pharmaceutical and has antiandrogenic activity.

This application claims the benefit of priority under 35 U.S.C. § 119(e) of U.S. Provisional Application No. 60/413,765, filed Sep. 27, 2002, and U.S. Provisional Application No. 60/470,223, filed May 14, 2003, the entire contents of each Application being incorporated herein by reference.

### BACKGROUND OF THE INVENTION

The present invention relates to new forms of bicalutamide, to compositions and pharmaceuticals containing the same, and to methods of making and using the foregoing.

Bicalutamide is the common name for the compound 4′-cyano-3-((4-fluorophenyl)sulfonyl)-2-hydroxy-2-methyl-3′-(trifluoromethyl)propionanilide, and is represented by the formula (1):

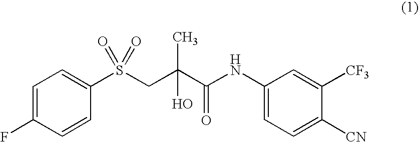

This compound can also be named N-(4-cyano-3-trifluoromethylphenyl)-3-(4-fluorophenylsulfonyl)-2-hydroxy-2-methyl-propionamide (see for instance J. Med. Chem. 31, 954-959 (1988) for the former nomenclature and WO 01-00608 for the latter nomenclature).

Bicalutamide and related acylanilides have been disclosed in EP 100172 and corresponding U.S. Pat. No. 4,636,505 as pharmaceutically active compounds that possess antiandrogenic activity. Such compounds are useful, inter alia, in treating prostate cancer. A bicalutamide pharmaceutical product is approved in many countries of the world under the brand name CASODEX (AstraZeneca). In marketed pharmaceutical compositions, bicalutamide is used as a racemate.

Bicalutamide is known to be isolated in a crystalline solid state. For example, U.S. Pat. No. 4,636,505 and the above-mentioned J. Med. Chem. article disclose that after synthesis of the compound, the solvent is evaporated, and the solid residue is crystallized from ethyl acetate/petroleum ether. Similarly, WO 01-00608 discloses that raw bicalutamide is recrystallized from a mixture of ethyl acetate and petroleum ether.

While the known bicalutamide is a stable crystalline form, it would be desirable to find other stable forms of bicalutamide.

### SUMMARY OF THE INVENTION

It has surprisingly been discovered that bicalutamide can be formed in a different polymorphic form than the known one. Thus, a first aspect of the present invention relates to a crystalline bicalutamide of form II. Typically form II bicalutamide can be distinguished from form I by an x-ray powder diffraction peak at about 25.9° or an IR absorbance peak at 847 cm⁻¹+/−5 cm⁻¹. A substantially pure bicalutamide form II exhibits an x-ray powder diffractogram substantially as shown in FIG. 2, as set forth hereinafter and an IR absorbance spectrum substantially corresponding to FIG. 4, as set forth hereinafter.

A further aspect of the present invention relates to the use of bicalutamide form II in making a medicament and in treating mammals in need of antiandrogenic effect. For example, a pharmaceutical composition comprising form II bicalutamide and a pharmaceutically acceptable excipient. In some embodiments a combination of bicalutamide form I and form II is used. In others, the composition is substantially free of bicalutamide form I.

A still further aspect of the present invention relates to a process for making bicalutamide form II, which comprises precipitating bicalutamide form II from a solution of bicalutamide. The precipitation can be carried out in the presence of seed crystals of bicalutamide form II and is usually induced or carried out by lowering the temperature of the bicalutamide solution and/or contacting the bicalutamide solution with a contrasolvent. Preferably the precipitation occurs at a temperature of 35° C. or higher. The bicalutamide form II can also be made by a process which comprises heating an amorphous bicalutamide to form one or more crystals of bicalutamide form II.

The amorphous bicalutamide is another aspect of the present invention. It can be formed by heating a solid form of bicalutamide to form a melt and cooling the melt to form amorphous bicalutamide.

### BRIEF DESCRIPTION OF THE DRAWINGS

FIG. 1 shows the XRPD of conventional bicalutamide form I.

FIG. 2 shows the XRPD of the novel bicalutamide form II produced in example 1.

FIG. 3 shows the IR absorbance spectrum for conventional bicalutamide form I.

FIG. 4 shows the IR absorbance spectrum for the novel bicalutamide form II produced in example 1.

FIG. 5 shows the IR absorbance spectrum for the novel bicalutamide form II produced in example 2.

FIG. 6 shows the DSC scan for conventional bicalutamide form I.

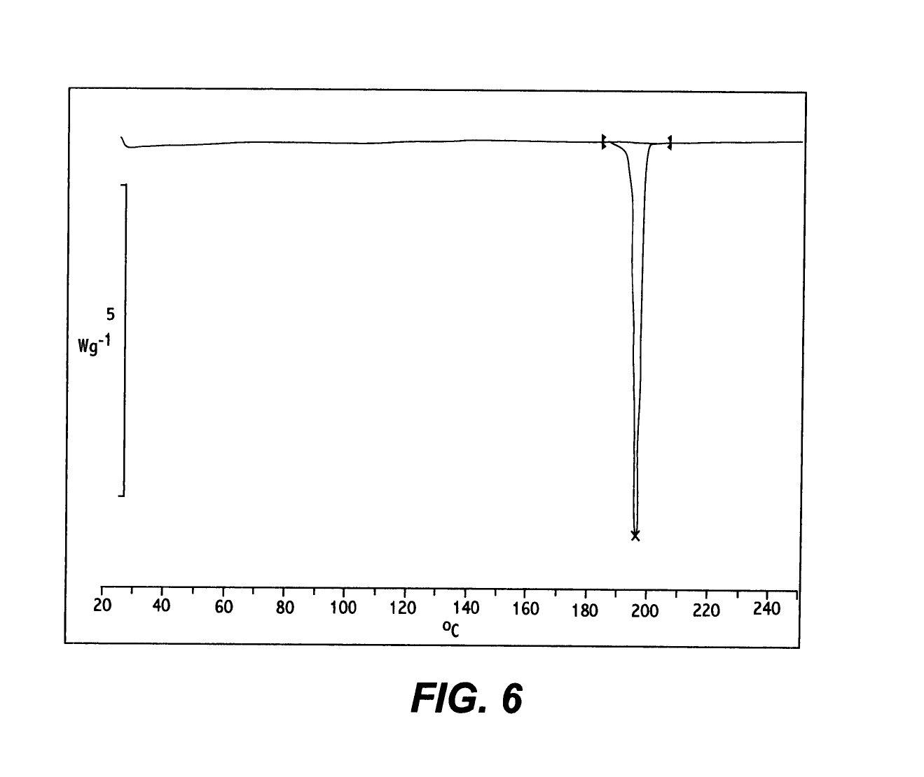

FIG. 7 shows the DSC scan for the novel bicalutamide form II produced in example 1.

FIG. 8 shows the DSC scan for the novel bicalutamide form II produced in example 2.

### DETAILED DESCRIPTION OF THE INVENTION

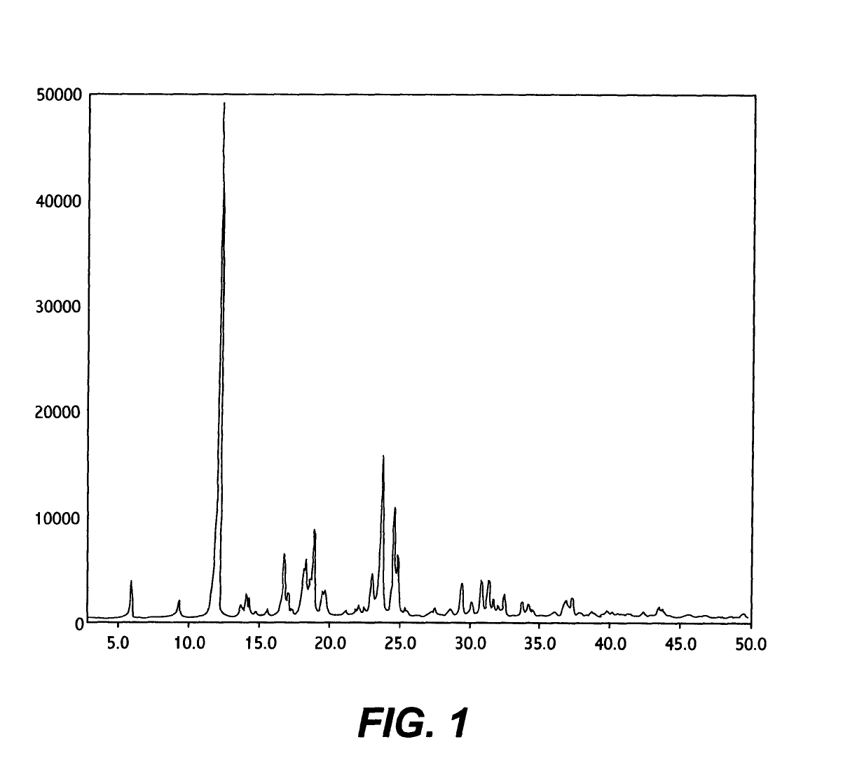

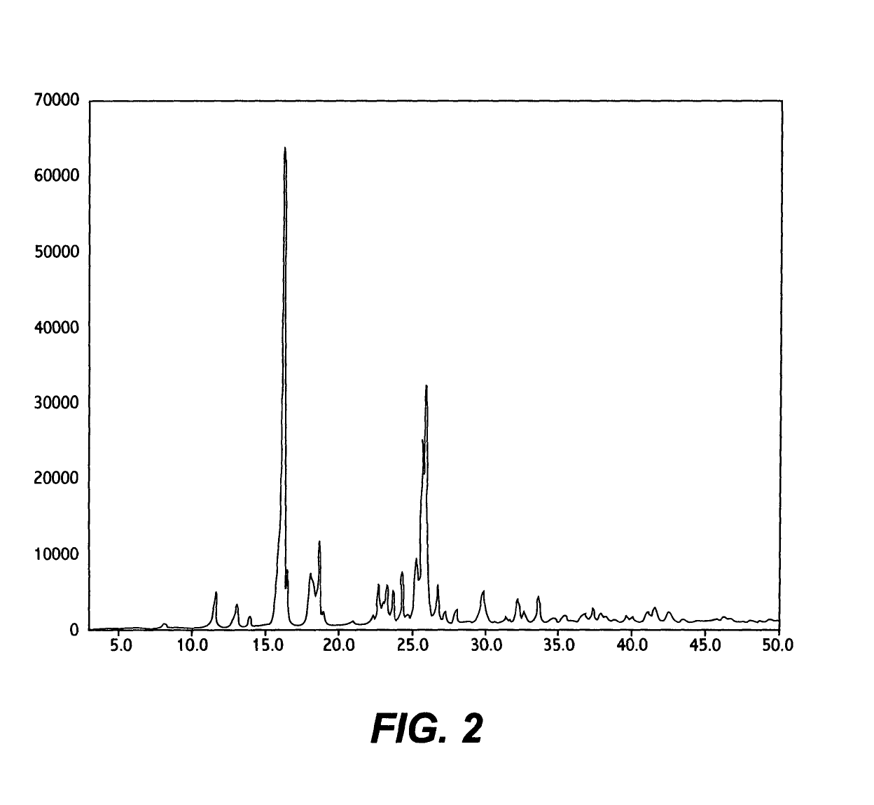

As a result of research investigations, a new polymorphic form of bicalutamide has been unexpectedly discovered. The known bicalutamide crystalline solid (referred to herein as “form I”) exhibits an x-ray powder diffraction (“XRPD”) pattern as shown in FIG. 1. However, the crystalline bicalutamide produced in example 1, hereinafter described, exhibits a different XRPD pattern from the known bicalutamide, as shown in FIG. 2. This difference in diffraction pattern indicates that the bicalutamide crystal can be arranged in different ways, i.e. different spatial arrangement of the bicalutamide molecules in the crystal lattice. This novel crystalline structure of the bicalutamide molecules is referred to herein as “form II.”

A particularly clear distinction in the two forms is seen at an angle of about 25.9°, more typically 25.85°+/−0.05°, in that bicalutamide form II exhibits a large peak while bicalutamide form I exhibits no peak. Thus, the presence of a peak at around 25.9°, especially at 25.85°+/−0.05°, can be used to characterize or identify the presence of the bicalutamide form II crystal structure in a bicalutamide sample. More generally, appreciable and/or large peaks in the XRPD pattern of bicalutamide form I are present at an angle (2θ) of about 6.2°, 9.6°, 12.4°, 14.3-14.6°, 17.0-17.4°, 19.7-20.1°, 24° and 31°, but such appreciable and/or large peaks are not present in the XRPD pattern of bicalutamide form II. On the other hand, the XRPD pattern of bicalutamide form II includes appreciable and/or large peaks at one or more angles (2θ) of about 11.6°, 13.0°, 16.2°, 18.1°, 24.4°, 25.3-25.9° (generally three peaks: 25.3, 25.6 and 25.9), 26.7°, 29.9° and 33.6°, while bicalutamide form I does not show appreciable and/or large peaks at these angles. In particular, a bicalutamide that exhibits an XRPD pattern that substantially corresponds with FIG. 2 is a specific embodiment of the present invention. The above-mentioned distinctions can be seen in comparing FIG. 1 (bicalutamide form I) with FIG. 2 (bicalutamide form II). Generally, the measured angle values for bicalutamide form I and form II are within +/−0.1° of the above-recited values, more preferably the measured values are identical to the above values after truncating or rounding.

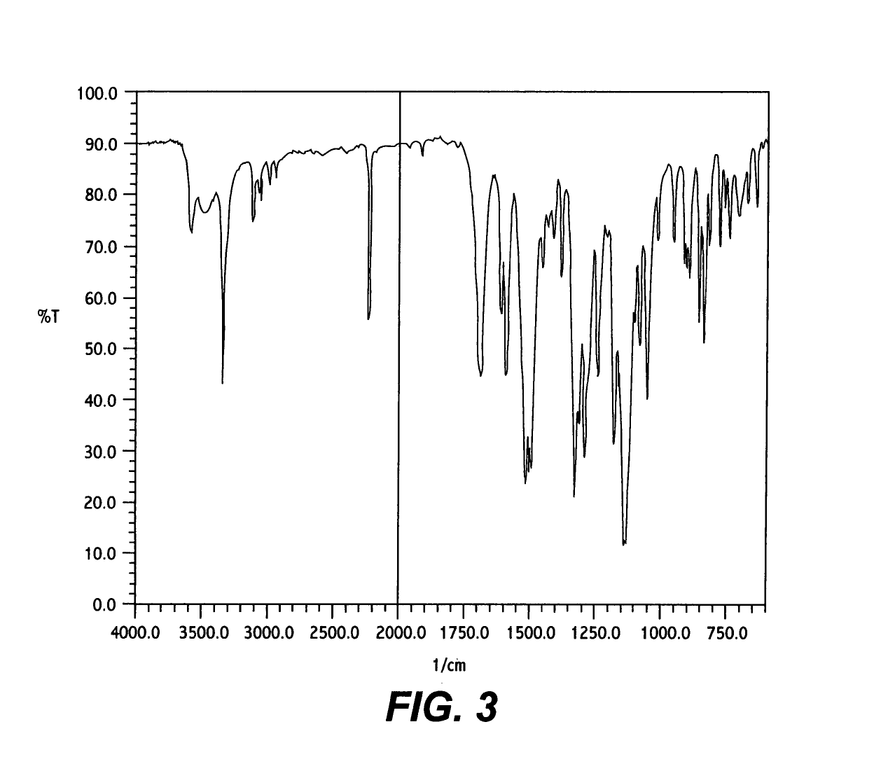

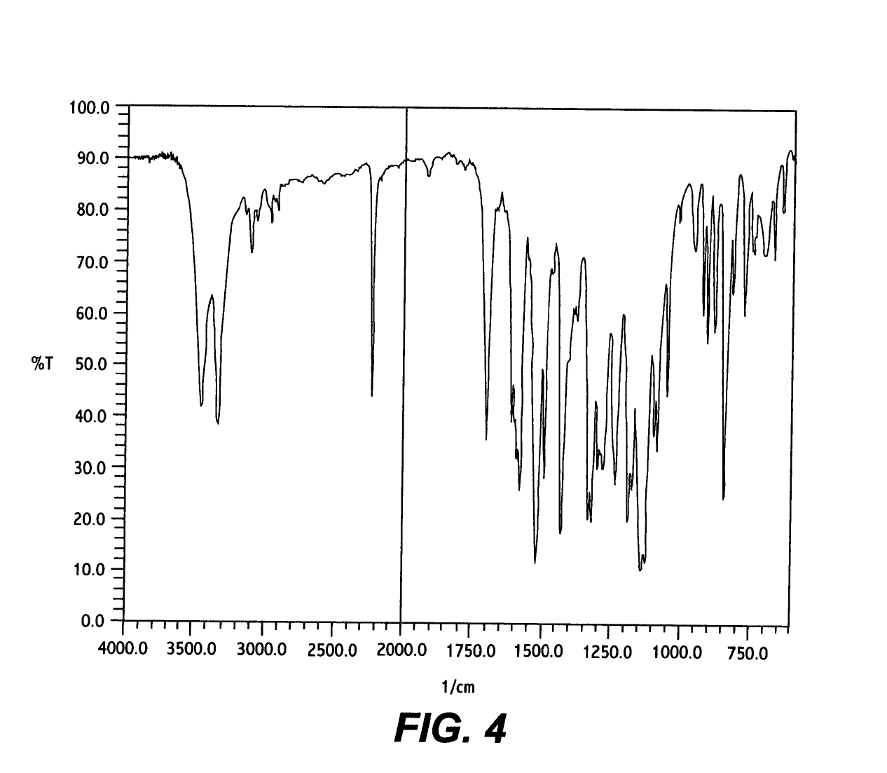

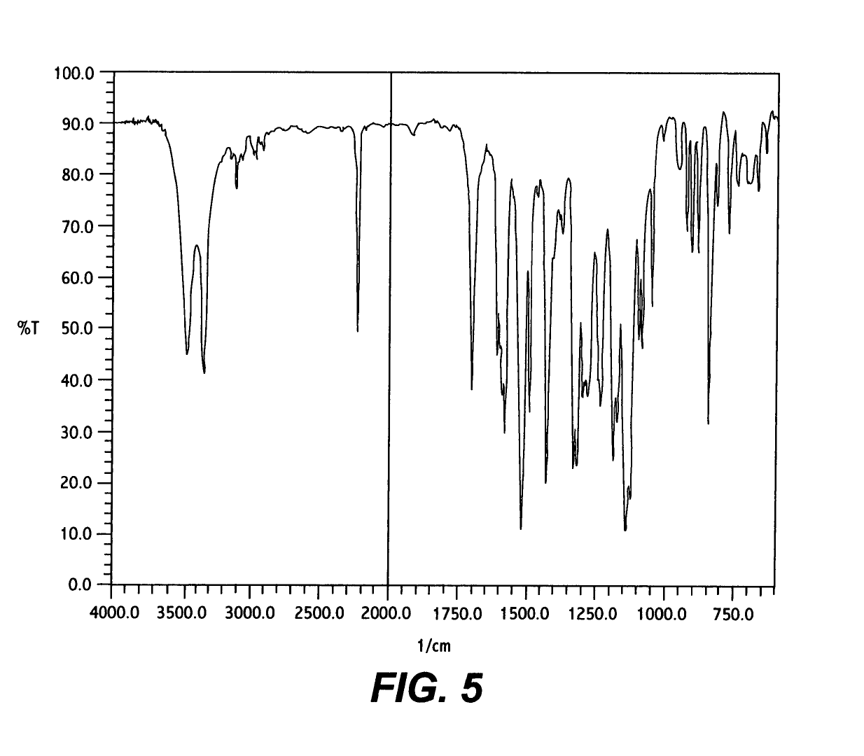

Somewhat surprisingly, the IR spectrum for the two forms is also different. As seen in comparing either FIG. 4 or 5 with FIG. 3, bicalutamide form II exhibits many differences in IR absorbance from bicalutamide form I, respectively. The most pronounced differences are observed between 3400-3600 cm⁻¹, around 1580 cm⁻¹, between 1495-1505 cm⁻¹, 1280-1450 cm⁻¹, 1175-1200 cm⁻¹ and 840-925 cm⁻¹. In particular, bicalutamide form II has a unique IR absorbance peak at about 847 cm⁻¹, while bicalutamide form I contains a doublet at 841 cm⁻¹ and 860 cm⁻¹. Thus, the presence of a peak at 847 cm⁻¹+/−5 cm⁻¹, preferably +/−3 cm⁻¹ can be used to characterize or identify the presence of the bicalutamide form II crystal structure in a bicalutamide sample. Similarly, a bicalutamide that exhibits an IR absorbance spectra that substantially corresponds to FIG. 4 is a specific embodiment of the present invention. The phrase “substantially corresponds” is used to allow for variations caused by different sample preparations, different equipment and/or settings used in measuring, normal experimental error/variation and small amounts of impurities. Differences in a pattern or spectra that are not attributable to these factors indicate that the pattern or spectra in question does not substantially correspond to the pattern of FIG. 2 or the spectra of FIG. 4, respectively. For example, the spectra for the example 2 material, shown in FIG. 5, substantially corresponds to the spectra in FIG. 4, even though it is not an identical, superimposable image. As is readily apparent to workers skilled in the art of comparing IR absorbance spectra, FIGS. 4 and 5 show the “same” spectra. The identification of bicalutamide form II is not limited to x-ray powder diffraction or IR spectra. Any technique that can distinguish the two forms such as by different physical properties can be used.

The present invention includes bicalutamide form II as an isolated substance, especially in a relatively pure form. “Relatively pure” means at least 70% pure, preferably at least 80% pure, more preferably at least 90% pure, still more preferably at least 95% including at least 98% pure, 99% pure, and at least 99.8% pure. The present invention also includes mixtures of bicalutamide form II with other forms of bicalutamide, especially with bicalutamide form I and/or amorphous bicalutamide. Thus, a composition that contains a small amount or a large amount of bicalutamide form II, regardless of the other materials/substances optionally present therewith, is contemplated to be part of the present invention.

The bicalutamide molecule can be made by synthesis techniques well known in the prior art, including the processes mentioned in the above-identified patents. The bicalutamide molecule contains one asymmetric carbon atom, thus allowing for the existence of both single enantiomers and a racemate. Preferably the bicalutamide used in the present invention is racemic and/or a mixture of enantiomers.

Bicalutamide form II may be obtained by precipitating crystalline bicalutamide of form II from a solution containing bicalutamide. The bicalutamide solution comprises a solvent and bicalutamide dissolved (including partly dissolved) therein. The solvent need only be capable of dissolving the bicalutamide under the conditions employed, e.g. temperature, concentration, etc. Suitable solvents include polar organic solvents such as alcohols, acids, and esters. Preferred solvents are ethyl acetate, methanol and ethanol. In one embodiment, the bicalutamide solution from which bicalutamide form II is precipitated is the solution resulting from the synthesis of bicalutamide.

The step of precipitating usually includes at least one of (1) reducing the temperature of the bicalutamide solution, (2) reducing the volume of the solvent in the bicalutamide solution, or (3) contacting the bicalutamide solution with a contrasolvent. The precipitation can be carried out in the presence of a bicalutamide form II seed crystal, but such is not required. Crystalline bicalutamide of form II is generally precipitated from the bicalutamide solution at a higher temperature than form I, although lower and/or comparable temperatures can be used when the precipitation is carried out in the presence of a form II seed crystal. Preferably, the precipitation occurs at a temperature of at least 30° C., preferably at least 35° C., and more preferably at least 40° C. If the precipitation is not spontaneous at the desired temperature, it is preferred to contact the solution with a contra-solvent to bring about precipitation. Preferred contra-solvents are petroleum ethers, especially those having a boiling point between 40° C. and 60° C. How the contacting is achieved is not particularly limited and includes adding the contrasolvent into the bicalutamide solution as well as adding the bicalutamide solution into the contrasolvent among others.

In a preferred embodiment, a suspension of seed crystals of bicalutamide form II in a suitable liquid carrier, such as hexane, heptane, cyclohexane, petroleum ether or mixtures thereof, is contacted with a bicalutamide solution wherein the solvent is at least partly miscible with the liquid carrier. Typically, a hot concentrated bicalutamide solution (preferably at a temperature from about 30° C. to about the reflux temperature) is contacted with a cold suspension (preferably of a temperature of about −20° C. to 20° C.) of the bicalutamide form II seed crystals. Crystals of form II are preferably formed at the temperature of contact and, optionally, the reaction mixture may be further cooled so that another portion of crystals may precipitate.

The obtained solid product may be separated from the liquid vehicle by any of the usual separation methods such as filtration or centrifugation, and may be optionally washed and dried. The dried product may be further milled and, optionally, sieved.

Another method for making bicalutamide form II comprises heating an amorphous bicalutamide to form one or more crystals of bicalutamide form II. The amorphous bicalutamide is generally heated to a melted or fluid state. The melting temperature is typically less than 175° C., more typically 160° C. or less. Bicalutamide form II crystallizes out of the “liquid.” Preferably the bicalutamide crystals are formed at a temperature between 150° C. and 175° C. such as 160° C.

The amorphous bicalutamide of the present invention can be formed by melting a solid bicalutamide, especially crystalline bicalutamide such as form I, and then cooling the melt to form an amorphous bicalutamide. Interestingly, the initial solid bicalutamide generally has a melting point that is higher than the melt or liquefying point for the resulting amorphous bicalutamide. The amorphous bicalutamide, which can also be considered a glass, does not have a true melting point; i.e. no distinctive peak under differential scanning calorimetry (DSC) analysis. In forming the amorphous bicalutamide, the melt is cooled typically by removing the heating source and allowing the material to cool under ambient and/or room temperature, although forced cooling or refrigeration may also be employed if desired. The solidified amorphous material can be isolated, ground/milled, and/or sieved if desired. The amorphous bicalutamide can be used, with or without isolation, to form crystalline bicalutamide of form II by heating as described above. The amorphous bicalutamide can be mostly converted to bicalutamide form II crystals, although complete conversion is not required.

Bicalutamide form II can be formulated into various pharmaceutical compositions with one or more pharmaceutically acceptable excipients. The pharmaceutical composition can be a unit dosage form such as a solid oral dosage form (i.e. tablet or capsule), a solution or suspension, especially for an aqueous sterile solution or suspension for parenteral administration, or bulk precursor thereof such as a pre-blended mixture ready for further blending/addition of ingredients, or a blend ready for tabletting or filling into capsules.

Usually the excipient is a pharmaceutically acceptable carrier or diluent such as one or more calcium phosphates, microcrystalline cellulose, hydroxypropyl methylcellulose, lactose, and starches, but is not limited thereto. In some embodiments, a polymer that is able to form a molecular dispersion with bicalutamide form II is used as an excipient. An example of such a polymer is hydroxypropylmethylcellulose phthalate. Such a dispersion can be formed by methods well known in the art; for example dissolving the active (bicalutamide form II in this invention) and the polymer in a suitable solvent and evaporating the solvent. Other excipients include fillers, binders, lubricants, disintegrants, preservatives, pH-adjustors, colorants, etc.

The pharmaceutical compositions are preferably formulated into tablets. The tablet may be monolithic tablets, i.e. tablets that upon ingestion do not disintegrate into a plurality of smaller units from which the active ingredient is finally released, or may be disintegrable tablets. The tablets may be produced by any standard tabletting technique, e.g. by wet granulation, dry granulation or direct compression. The tabletting methods that do not employ a solvent (“dry processes”) are preferable. The tablet compositions may be further coated by a film coat. The film coat may protect the tablet against the environment (light, air, moisture) during storage and handling. Any conventional film coat may be used.

Alternatively, bicalutamide pharmaceutical compositions can be filled into capsules. Generally, the process comprises blending the bicalutamide active substance and excipients in one or more mixing or blending steps and then filling the blend into capsules.

The pharmaceutical compositions of the present invention contain bicalutamide form II as either the only bicalutamide form or as one of two or more forms. Thus, in one embodiment, the pharmaceutical composition is substantially free of bicalutamide form I, i.e. contains less than 0.2%, more preferably less than 0.1%, preferably less than 0.01%. In another embodiment, the pharmaceutical composition contains a mixture of bicalutamides, such as bicalutamide form I and form II, wherein the relative amount of form II is within the range of 0.1% to 99.8%, based on the total weight of all forms of bicalutamide. Typically at least 1.0%, more typically at least 10%, and preferably at least 90% of the bicalutamide is bicalutamide form II.

The pharmaceutical composition of the present invention is normally formulated into a unit dosage form such as the above-described tablets or capsules. In a unit dosage form, the total amount of bicalutamide present, regardless of form, is effective for providing an antiandrogenic effect to a mammal. Typically the amount of bicalutamide is from 1 to 600 mg, more typically from 1 to 300 mg, preferably from 30 to 150 mg, such as 50 mg, 100 mg, and 150 mg doses. The unit dose may be a single tablet, one half of a tablet, or two or more tablets taken at essentially the same time or in the same administration. Unit dose in capsule form may comprise one or more capsules. In a particularly preferred embodiment, bicalutamide Form II can be formulated, as an active component, into the CASODEX tablet formulation that is commercially sold. That is, the bicalutamide form II is present as a replacement for some or all of the bicalutamide form I in the commercial tablet; all excipients and proportions remaining the same. Additional embodiments of pharmaceutical compositions, pharmaceutical dosage forms, and their preparation, which can be applied to formulating bicalutamide form II, are described in commonly owned, co-pending U.S. Provisional Patent Application No. 60/470,224, filed May 14, 2003, the entire contents of which are incorporated herein by reference.

The bicalutamide pharmaceutical composition can further contain another pharmaceutically active ingredient. Examples include progestins, luteinizing hormone-releasing hormone (LH-RH) or analogues thereof, an aromatase inhibitor, antibiotics, or anti-inflammatory agents.

The bicalutamide containing at least a portion of bicalutamide form II (i.e., at least 0.1%) can be used to treat a mammal in need thereof by administering an antiandrogenic effective amount of the bicalutamide. The effective amount is generally within the range of 0.1 to 125 mg/kg of body weight. Typically the amount administered is from 1 to 600 mg, more typically from 1 to 300 mg and especially 50, 100 or 150 mg in the form of one or two tablets or capsules.

Additional pharmaceutically active ingredients can be co-administered with the bicalutamide. For example, progestins, luteinizing hormone-releasing hormone (LH-RH) or analogues thereof, an aromatase inhibitor, antibiotics, or anti-inflammatory agents can be administered concurrently with, simultaneously with, or in the same pharmaceutical composition as the bicalutamide. A preferred regimen for treating prostate cancer is the use of bicalutamide once a day at 150 mg with goserelin. The goserelin can be orally administered or continuously supplied by implant.

The present invention is further described by the following non-limiting examples.

### EXAMPLES

### Reference Example: Bicalutamide form I

2.15 g of bicalutamide and 19.5 ml of ethyl acetate were transferred into a round bottomed 3 neck flask of 250 ml. The suspension was heated to reflux in an oil bath and stirred with magnetic stirrer and stirrer device. Reflux was maintained until a clear solution was obtained. The solution was cooled to 20° C. in a water bath while kept stirring. During cooling the bicalutamide crystallized. The suspension was then cooled to 5° C. in an ice bath. To the suspension 77 ml of petroleum ether (boiling range 40-70° C.) was added slowly. After addition, the suspension was stirred for 5 more minutes. The suspension was filtered over a p3-glass filter using reduced pressure. The solid material was washed with cold petroleum ether (boiling range 40-70° C.). The solid material was then dried at 60° C. and under vacuum overnight. According to DSC, IR and microscopy the obtained bicalutamide is crystalline form I. The XRPD included the following peaks:

2θ (degrees)

6.225
9.595
12.370
14.370
14.610
17.060
17.370
19.710
20.110
23.970
24.775
25.080

### Example 1

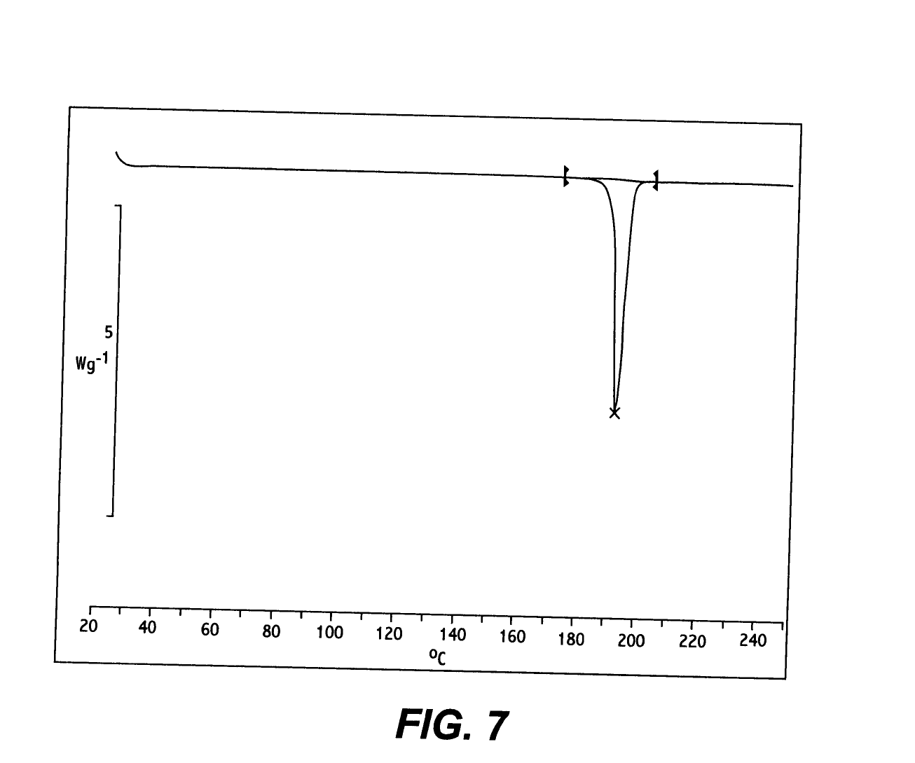

1.0 g of bicalutamide form I was transferred into a glass round bottomed flask of 100 ml. The flask was closed with a stopper and placed in an oil bath at 210° C. Within 5 minutes all active substance was molten (light yellow melt). Subsequently the flask was removed from the oil bath and the melt was allowed to cool to ambient temperature. The melt solidified to a glass. The flask was placed in an oil bath at 160° C. Within a few minutes the glass became liquid and crystals of bicalutamide form II were formed. The flask was removed from the oil bath after about 10 minutes and allowed to cool to ambient temperature. The solid mass was isolated and gently grinded to obtain particles, small enough for analysis. HPLC and NMR showed no degradation. The DSC, set forth is FIG. 7, shows a melting peak at 192.4° C. with onset at 190.6° C. The XRPD pattern is shown in FIG. 2 and includes the following peaks:

2θ (degrees)
11.645
13.065
16.215
18.080
24.355
25.265
25.630
25.865
26.750
29.885
33.605

The IR absorbance spectrum is shown in FIG. 4.

### Example 2

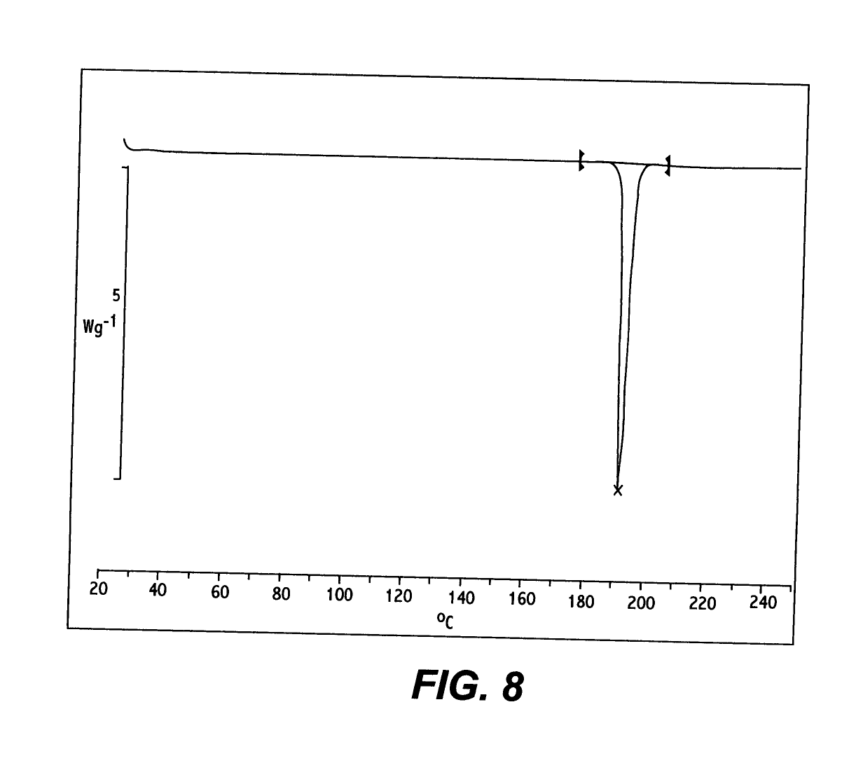

About 1-5 mg of the bicalutamide form II obtained in example 1 was suspended in 7 ml n-heptane (in a round bottomed flask of 100 ml). The flask was placed in a water-ice bath and the suspension was stirred with a magnetic stirrer and stirrer device. 0.5 gram of bicalutamide form I was dissolved in 7 ml ethyl acetate at reflux (in a round bottomed flask of 100 ml). The warm solution was added dropwise to the stirred cold heptane suspension using a warm glass capillary pipette. During the addition there was immediate precipitation of fine, white particles and finally a milky suspension was obtained. After a few minutes stirring, the suspension was filtered over a p3-glass filter using reduced pressure. The solid material was washed with a few ml n-heptane. The solid material was dried at ambient temperature and under vacuum for 1.5 hours. The DSC, shown in FIG. 8, shows a melting peak at 191.6° C. with onset at 191.0° C. The IR absorbance spectrum is shown in FIG. 5.

### Example 3

In a 10 l round-bottomed flask equipped with a mechanical stirrer, nitrogen inlet and a ice-acetone cooling bath, 3 l of n-heptane was cooled to −5 to −10° C. 190 g of bicalutamide was dissolved in 2.52 l of ethyl acetate at reflux. The cold, stirred n-heptane was seeded with 200 mg of bicalutamide form II obtained from Example 2. The hot bicalutamide solution in ethyl acetate was added slowly in 30 minutes to the cold stirred and seeded n-heptane. A white suspension was formed. The white suspension was stirred for 5 minutes and filtered over a glass-filter. Filtration took about 40 minutes. The white solid was washed with 2×200 ml cold n-heptane (0-4° C.). The solid was dried at air for 3 hours and was dried under vacuum at room temperature for 16 hours. Yield: 160 g of bicalutamide Form II; m.p. 189.7-191.6° C.; DSC: Tₒₙₛₑₜ 190.6° C. and Tₚₑₐₖ 191.2° C.; LOD: 0.1%; Purity: 99.78% (HPLC). The XRPD included the following peaks:

2θ (degrees)
11.555
12.990
16.150
18.110
24.300
25.195
25.570
25.800
26.685
29.870
33.610

### Example 4

1.0 g of bicalutamide and 25 ml ethyl acetate were transferred into a round bottomed 3 neck flask of 250 ml. The suspension was stirred (magnetic stirrer and stirrer device) and refluxed (oil bath) until a clear solution was obtained. Reflux was then maintained for 5 more minutes. The solution was cooled down to 40° C. Subsequently 100 ml of petroleum ether (boiling range 40-70° C.) was added dropwise to the stirred solution. During the addition, precipitation/crystallization of bicalutamide took place. After addition the suspension was cooled down to room temperature, while kept stirring. The suspension was filtered over a p3-glass filter using reduced pressure. The residue was washed with cold petroleum ether (boiling range 40-70° C.). The solid material was then divided into two portions. One portion was dried at room temperature and under vacuum overnight. The other portion was dried at 60° C. and under vacuum for 3 hours. According to DSC, IR, and X-ray, both portions are present as pure form II. The drying temperature did not have any effect on the present crystalline form. The XRPD included the following peaks:

2θ (degrees)
11.585
13.050
16.190
18.150
24.350
25.255
25.610
25.830
26.730
29.910
33.645

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

We Claim:

1. A crystalline bicalutamide of form II.

2. The bicalutamide according to claim 1, having an IR absorbance peak at 847 cm⁻¹+/−5 cm⁻¹.

3. The bicalutamide according to claim 1, having an IR absorbance spectra substantially as shown in FIG. 4.

4. The bicalutamide according to claim 1, having an x-ray diffraction peak at an angle of about 25.9°.

5. The bicalutamide according to claim 4, having an x-ray diffraction peak at an angle of 25.85°+/−0.05°.

6. The bicalutamide according to claim 1, having x-ray diffraction peaks at angles of about 11.6°, 13.0°, 18.1°, 24.4°, 25.3-25.9°, 26.7°, 29.9° and 33.6°.

7. The bicalutamide according to claim 1, having x-ray diffraction peaks at angles of about 11.6°, 13.0°, 16.2°, 18.1°, 24.4°, 25.3-25.9°, 26.7°, 29.9° and 33.6°.

8. The bicalutamide according to claim 1, wherein said bicalutamide has an x-ray diffractogram substantially as shown in FIG. 2.

9. The bicalutamide according to claim 1, wherein said bicalutamide is racemic bicalutamide.

10. The bicalutamide according to claim 1, wherein said bicalutamide is at least 90% pure form II bicalutamide.

11. Crystalline bicalutamide having an x-ray diffraction peak at an angle of about 25.9°.

12. A bicalutamide in amorphous form.

13. A composition comprising crystalline bicalutamide of form II and at least one of crystalline bicalutamide of form I and amorphous bicalutamide.

14. A pharmaceutical composition comprising the bicalutamide of form II according to claim 1, and a pharmaceutically acceptable excipient.

15. The pharmaceutical composition according to claim 14, wherein said composition is a unit dose and said bicalutamide of form II is contained in an antiandrogenic effective amount.

16. The pharmaceutical composition according to claim 15, wherein said composition is substantially free of form I bicalutamide.

17. The pharmaceutical composition according to claim 14, wherein said pharmaceutically acceptable excipient is a carrier or diluent.

18. The pharmaceutical composition according to claim 17, wherein said excipient is selected from the group consisting of calcium phosphates, microcrystalline cellulose, hydroxypropyl methylcellulose, lactose, and starches.

19. The pharmaceutical composition according to claim 14, wherein said composition is a solid oral dosage form.

20. The pharmaceutical composition according to claim 14, wherein said composition is a solution or suspension.

21. The pharmaceutical composition according to claim 14, which further comprises bicalutamide form I.

22. The pharmaceutical composition according to claim 21, wherein the relative amount of bicalutamide form II is within the range of 0.1-99.9% based on the total weight of all forms of bicalutamide.

23. A method for producing an antiandrogenic effect, which comprises administering an antiandrogenic effective amount of the bicalutamide according to claim 1 to a mammal in need thereof.

24. A process, which comprises precipitating bicalutamide form II from a solution containing bicalutamide.

25. The process according to claim 24, wherein said precipitation is carried out in the presence of seed crystals of bicalutamide form II.

26. The process according to claim 24, wherein said precipitation is carried out by lowering the temperature of the bicalutamide solution and/or contacting said bicalutamide solution with a contrasolvent.

27. The process according to claim 24, wherein said precipitation occurs at a temperature of 35° C. or higher.

28. A process which comprises heating an amorphous bicalutamide to form one or more crystals of bicalutamide form II.

29. A process which comprises heating a solid form of bicalutamide to form a melt and cooling said melt to form amorphous bicalutamide.
